(19) United States
(12) Patent Application Publication
(10) Pub. No.: US 2017/0315110 A1
(43) Pub. Date: Nov. 2, 2017

(54) SYSTEMS AND METHODS FOR PERSONALIZED SAMPLE ANALYSIS

(71) Applicant: The Trustees of Princeton University, Princeton, NJ (US)

(72) Inventor: Stephen Y. Chou, Princeton, NJ (US)

(21) Appl. No.: 15/520,398

(22) PCT Filed: Oct. 20, 2015
(86) PCT No.: PCT/US2015/056518

§ 371 Date: Apr. 19, 2017

Related U.S. Application Data
(60) Provisional application No. 62/066,777, filed on Oct. 21, 2014.

(51) Int. Cl. G01N 33/487; G01N 21/64

(52) U.S. Cl. CPC G01N 33/48792; G01N 21/6428; G01N 2021/6439

(57) ABSTRACT

Systems and methods for monitoring the health status of a subject are provided. In certain embodiments, the method includes: applying a sample provided from a subject to a signal enhancing detector configured to indicate an output that is representative of the sample; processing the output with a device configured to acquire the detector output as input data and process the input data to generate a report; and receiving the report. In certain embodiments, the device is a mobile device. In certain embodiments, the method further includes transmitting the sample-derived data in the device to a remote location where the transmitted data is analyzed; and receiving the results of the analysis. Also provided are systems for use in practicing the methods. Kits for use in monitoring the health status of a subject are also provided.

### CROSS-REFERENCING

This application claims the benefit of provisional application Ser. No. 62/066,777, filed Oct. 21, 2014 (NSNR-014PRV);

this application is also a continuation-in-part of U.S. application Ser. No. 13/838,600, filed Mar. 15, 2013 (NSNR-003), which application claims the benefit of U.S. provisional application Ser. No. 61/622,226 filed on Apr. 10, 2012, and is a continuation-in-part of U.S. patent application Ser. No. 13/699,270, filed on Jun. 13, 2013, which application is a §371 filing of US2011/037455, filed on May 20, 2011, and claims the benefit of U.S. provisional application Ser. No. 61/347,178, filed on May 21, 2010;

this application is also a continuation-in-part of U.S. application Ser. No. 14/431,266, filed on Mar. 25, 2015 (NSNR-002), which application is a §371 filing of international application serial no. PCT/US13/62923, filed on Oct. 1, 2013, which application claims the benefit of U.S. Provisional Patent Application Ser. No. 61/708,314, filed on Oct. 1, 2012;

this application is also a continuation-in-part of U.S. application Ser. No. 13/699,270, filed Jun. 13, 2013 (NSNR-001), which application is a §371 filing of international application serial no. US2011/037455, filed on May 20, 2011, which application claims the benefit of U.S. Provisional Patent Application Ser. No. 61/347,178 filed on May 21, 2010;

this application is also a continuation-in-part of U.S. application Ser. No. 14/775,634, filed Sep. 11, 2015 (NSNR-004), which application is a §371 filing of international application serial no. US2014/029979, filed on Mar. 15, 2014, which application claims the benefit of U.S. Provisional Patent Application Ser. No. 61/801,424, filed Mar. 15, 2013;

this application is also a continuation-in-part of U.S. application Ser. No. 14/775,638 filed Sep. 11, 2015 (NSNR-005), which application is a §371 filing of international application serial no. US2014/028417, filed on Mar. 14, 2014, which application claims the benefit of U.S. Provisional Patent Application Ser. No. 61/801,096, filed Mar. 15, 2013;

this application is also a continuation-in-part of U.S. application Ser. No. 14/852,417, filed Sep. 11, 2015 (NSNR-006), which application is continuation of international application serial no. US2014/029675, filed on Mar. 14, 2014, which application claims the benefit of U.S. Provisional Patent Application Ser. No. 61/800,915, filed Mar. 15, 2013;

this application is also a continuation-in-part of U.S. application Ser. No. 14/852,412, filed Sep. 11, 2015 (NSNR-0010), which application is a continuation of international application serial no. US2014/030108, filed on Mar. 16, 2014, which application claims the benefit of U.S. Provisional Patent Application Ser. No. 61/794,317, filed Mar. 15, 2013;

all of which applications are incorporated by reference herein for all purposes.

### INTRODUCTION

Providing simple access to an individual's biomarker status using personal monitoring systems and methods will be helpful for early detection of the onset of disease or monitoring of everyday changes in health status. Various biomarkers for disease and health status are known and their number is growing rapidly. However, conventional methods for monitoring the health of an individual using biomarkers requires invasive sample collection procedures, a specialized sample handling facility for sample collection and processing, bulky and costly assay readers, and technical staff to analyze the samples, making the process time consuming, intrusive and expensive. Thus, there is a need for fast, non-invasive and cost-effective ways to determine the health of an individual.

### SUMMARY

Systems and methods for monitoring the health status of a subject are provided. In certain embodiments, the method includes: applying a sample provided from a subject to a signal enhancing detector configured to indicate an output that is representative of the sample; processing the output with a device configured to acquire the detector output as input data and process the input data to generate a report; and receiving the report. In certain embodiments, the device is a mobile device. In certain embodiments, the signal enhancing detector is a microfluidic device. The signal enhancing nature of the detectors offers many advantages in detection speed, reduced sample volume, reduced reagent usage, simple signal readers, non-invasiveness, and low cost. In some embodiments, the microfluidic device includes a nanosensor. In certain embodiments, the method further includes transmitting the sample-derived data in the device to a remote location where the transmitted data is analyzed; and receiving the results of the analysis.

Also provided are systems for use in practicing the methods. The system may include a device configured to: acquire as input data output from a signal enhancing detector; process the input data to generate a report; and provide the report to the subject, wherein the signal enhancing detector is configured to obtain a sample provided from the subject and indicate an output that is representative of the sample. In certain embodiments, the device is configured to transmit the sample-derived data to a remote location where the transmitted information is analyzed. In certain embodiments, the device is configured to receive the results of the analysis, and provide the analyzed results to the subject. Kits for use in monitoring the health status of a subject are also provided.

### BRIEF DESCRIPTION OF THE FIGURES

FIG. 1 depicts a schematic representation of a mobile device configured to acquire an output from a signal enhancing detector, according to embodiments of the invention. Panels A-C illustrate an embodiment of a D2PA array.

FIG. 2 depicts a schematic representation of the personal health monitoring system and its use, according to embodiments of the invention.

FIG. 3 depicts a signal enhancing detector that includes a microfluidic nanosensor, according to embodiments of the invention.

FIG. 4 depicts a schematic representation of a disk-coupled dots-on-pillar antenna array (D2PA) signal enhancing detector and an amyloid beta immunoassay using the same, according to embodiments of the invention.

FIG. 5 shows immunoassay standard curves for different biomarkers on D2PA, according to embodiments of the invention.

FIG. 6 shows monitoring of salivary beta amyloid 1-42 levels in healthy human subjects, according to embodiments of the invention.

FIG. 7 shows a “box-diagram” illustrating the relative position of each “layer”. The diagram is not in scale, nor reflects the fact some “layers” of discrete molecules. The molecular adhesion layer is optional.

### DETAILED DESCRIPTION

As summarized above, an aspect of the invention is directed to a method for monitoring the health status of a subject, the method including: applying a sample provided from a subject to a signal enhancing detector configured to indicate an output that is representative of the sample; processing the detector output with a device configured to acquire the detector output as input data and to analyze the input data to generate a report; and receiving the report. The signal enhancing detector offers the advantages of fast detection, simplified reader (e.g. replace large conventional reader by smartphone), and lost cost.

In certain embodiments, the signal enhancing detector includes a disk-coupled dots-on-pillar antenna array (D2PA), which have been described in U.S. application Ser. No. 13/838,600, filed Mar. 15, 2013 (NSNR-003) and other related applications listed in the cross-referencing paragraph set forth above, all of which applications are incorporated by reference herein for all purposes.

In some embodiments, the signal enhancing detectors use a different a “signal amplification layer” (SAL) other than the D2PA (namely the SAL replaces the D2PA), which have been described in U.S. Provisional application Ser. No. 61/794,317, filed Mar. 15, 2013 (NSNR-010PRV) and other related applications listed in the cross-referencing paragraph set forth above, all of which applications are incorporated by reference herein for all purposes. D2PA is only one example of a signal amplifying layer. Since, in some embodiments, the D2PA may be replaced by a different signal amplification layer, the present invention includes other embodiments of devices and methods in which the D2PA is replaced by another SAL, while other aspects of the devices, systems and methods may be are unchanged.

In some embodiments, the device provides an advice to the subject. In some embodiments, the method includes acquiring an output as input data from a signal enhancing detector, wherein the signal enhancing detector is configured to indicate an output that is representative of a sample provided by a subject, analyzing the input data, and delivering a report to the subject who holds the device or is in the same location of the device, and/or is in a remote location. In some embodiments, the report may include an advice for the subject. In certain embodiments, the method further includes: transmitting the sample-derived data in the device to a remote location where the transmitted information is analyzed; and receiving the results of the analysis. In certain embodiments, the report is related to health conditions of the subject. In certain embodiments, the analysis is done by either software or a professional. In certain embodiments, the analysis and advice of next actions will be provided to the subject.

Also provided is a system that includes a device configured to: acquire as input data an output from a signal enhancing detector; process the input data to generate a report; and provide the report to the subject, wherein the signal enhancing detector is configured to obtain a sample provided from the subject and indicate an output that is representative of the sample. In certain embodiments, the device is configured to transmit the sample-derived data to a remote location where the transmitted information is analyzed. In other embodiments, the device is configured to receive the results of the analysis and provide the analyzed results.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a,” “an” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Methods

Aspects of the invention are directed to a method for monitoring the health status of a subject, i.e., detecting levels of biomarkers in a sample provided from the subject and diagnosing the subject for a disease or predisposition thereof. The method in certain embodiments includes applying a sample provided from a subject to a signal enhancing detector to indicate an output, such as a nanoplasmonic-enhanced fluorescence signal, that is representative of the sample. The method may further include processing the detector output with a device, such as a mobile device, that acquires as input data the detector output and processes the input data to generate a report, which may be received by the subject, for example, in the form of a graph and/or a color-coded health status/recommended action indicator. In certain embodiments, the method includes transmitting the sample-derived data to a remote location, e.g., a hospital or other medical center, or a research institution, where a health care professional analyzes the transmitted data. The method may further include the subject receiving the analyzed data. The various aspects of the invention are now described in greater detail below.

### Signal Enhancing Detector

As summarized above, embodiments of the method include applying a sample provided from a subject to a signal enhancing detector configured to indicate an output that is representative of the sample. A signal enhancing detector according to embodiments of the present system may be any signal enhancing detector suitable for use in the subject methods. In certain embodiments, the signal enhancing detector is configured to detect the presence or absence of an analyte of interest in a sample. Analytes of interest include, but are not limited to, proteins, nucleic acids (DNA and RNA), lipids, carbohydrates, vitamins, hormones, synthetic hormone analogues, organic polymers, heavy metals, drugs, etc, and any metabolites thereof. Any suitable method of detecting an analyte of interest may be employed in the subject methods. In certain embodiments, detection may be achieved by specific binding of a binding agent to the analyte of interest. Binding agents of interest include, but are not limited to, antibodies (including antigen binding fragments thereof), nucleic acids (DNA, RNA), aptamers, lectins, enzymes, etc.

In some embodiments, the signal enhancing detector is configured to produce a signal in the presence of an analyte of interest in the sample. A signal produced by the signal enhancing detector may be in the form of a light emitted under external excitation, for example, fluorescence or luminescence, including electroluminescence and chemiluminescence. In certain cases, the signal may be produced by a signal-producing member. The signal-producing members of interest include, but are not limited to, fluorophores (e.g., xanthene dyes, e.g. fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′, 5′-dichloro-2′, 7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 1 10; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. Specific fluorophores of interest that are commonly used in subject applications include: Pyrene, Coumarin, Diethylaminocoumarin, FAM, Fluorescein Chlorotriazinyl, Fluorescein, R1 10, Eosin, JOE, R6G, Tetramethylrhodamine, TAMRA, Lissamine, ROX, Napthofluorescein, Texas Red, Napthofluorescein, Cy3, and Cy5, IRDye800, IRDye800CW, Alexa 790, Dylight 800, etc); chemiluminescent agents (e.g., acridinium esters and sulfonamides, luminol and isoluminol); and electrochemiluminescent agents (e.g., ruthenium (II) chelates).

In certain embodiments, the signal producing member is configured to bind specifically to the analyte of interest. In certain embodiments, a signal-producing member may be present in the signal enhancing detector before the sample is applied to the detector. In other embodiments, the signal-producing member may be applied to the detector after the sample is applied to the detector.

In certain embodiments, the signal is representative of the sample or a portion thereof. In certain embodiments, a signal representative of the sample is an intensity value of the light emitted under external excitation that is proportional to the amount of the analyte of interest that is present in the sample. In certain embodiments, the signal change is representative of the sample. Thus, a signal change representative of the sample may be a change in intensity of light emitted under external excitation that is proportional to the amount of analyte of interest that is present in the sample. The external excitation may be provided from any suitable source, including, but not limited to, sun light, ambient light, LEDs, lasers, etc. for fluorophores.

In certain aspects, a signal enhancing detector enhances the signal, e.g., fluorescence or luminescence, that is produced by the signal-producing member. In certain embodiments, the signal is enhanced by a physical process of signal amplification. In some embodiments, the signal is enhanced by a nanoplasmonic effect (e.g., surface-enhanced Raman scattering). Examples of signal enhancement by nanoplasmonic effects is described, e.g., in Li et al, Optics Express 2011 19: 3925-3936 and WO2012/024006, which are incorporated herein by reference. In certain embodiments, signal enhancement is achieved without the use of biological/chemical amplification of the signal. Biological/chemical amplification of the signal may include enzymatic amplification of the signal (e.g., used in enzyme-linked immunosorbent assays (ELISAs)) and polymerase chain reaction (PCR) amplification of the signal. In other embodiments, the signal enhancement may be achieved by a physical process and biological/chemical amplification.

In certain embodiments, the signal enhancing detector is configured to enhance the signal by 10³ fold or more, for example, 10⁴ fold or more, 10⁵ fold or more, 10⁶ fold or more, including 10⁷ fold or more, compared to a detector that is not configured to enhance the signal. In certain embodiments, the signal enhancing detector is configured to enhance the signal by 10³ fold or more, for example, 10⁴ fold or more, 10⁵ fold or more, 10⁶ fold or more, including 10⁷ fold or more, compared to a detector that is not configured to enhance the signal using a physical amplification process, as described above. In certain embodiments, the signal enhancing detector is configured to have a detection sensitivity of 0.1 nM or less, such as 10 pM or less, or 1 pM or less, or 100 fM or less, such as 10 fM or less, including 1 fM or less, or 0.5 fM or less, or 100 aM or less, or 50 aM or less, or 20 aM or less. In some instances, the signal enhancing detector is configured to be able to detect analytes at a concentration of 1 ng/mL or less, such as 100 pg/mL or less, including 10 pg/mL or less, 1 pg/mL or less, 100 fg/mL or less, 10 fg/mL or less, or 5 fg/mL or less. In certain embodiments, the signal enhancing detector is configured to have a dynamic range of 5 orders of magnitude or more, such as 6 orders of magnitude or more, including 7 orders of magnitude or more.

In certain embodiments, the signal enhancing detector is configured to indicate an output that is representative of the sample applied or a portion thereof. In certain aspects, the output indicated by the signal enhancing detector is an enhanced signal that is representative of the sample or a portion thereof. As such, in some instances, the indicated output is representative of the sample or a portion thereof. In certain aspects, an output representative of the sample may be an intensity of light emitted under external excitation that is proportional to the amount of the analyte of interest that is present in the sample. In certain aspects, an output representative of the sample may be a change in the intensity of light emitted under external excitation, from before to after the sample is applied, that is proportional to the amount of the analyte of interest that is present in the sample. In certain aspects, an output representative of the sample may be a difference in the intensity of light emitted under external excitation, between a sample obtained from a subject and a reference sample that contains a known amount of the analyte of interest, that is proportional to the amount of the analyte of interest that is present in the sample.

In certain embodiments, the signal enhancing detector includes a light source, such as LEDs, photodiodes or other light sources. In some embodiments the signal enhancing detector includes optical filters.

In certain embodiments, the signal enhancing detector is configured to convert the amplified signal to data. Thus, in certain embodiments, the signal enhancing detector includes sensors and components including photodiodes, photomultiplier tubes, photoelectric cells, and other light-sensitive electronic components that may be used to provide, in whole or in part, electronic data representative of the sample. In certain embodiments, the signal enhancing detector includes a camera, a luminometer, or a spectrophotometer.

Thus, in some embodiments, the signal enhancing detector indicates an output by emitting light under external excitation, as described above. In other embodiments, the signal enhancing detector output is data that is representative of the sample. In certain embodiments, the signal enhancing detector includes a memory, such as a memory chip and/or a microprocessor, in which to store the data. In certain embodiments, the signal enhancing detector is configured to communicate over a network.

In certain embodiments, a sample may include various fluid or solid samples. In some instances, the sample can be a bodily fluid sample from the subject. In some instances, solid or semi-solid samples can be provided. The sample can include tissues and/or cells collected from the subject. The sample can be a biological sample. Examples of biological samples can include but are not limited to, blood, serum, plasma, a nasal swab, a nasopharyngeal wash, saliva, urine, gastric fluid, spinal fluid, tears, stool, mucus, sweat, earwax, oil, a glandular secretion, cerebral spinal fluid, tissue, semen, vaginal fluid, interstitial fluids derived from tumorous tissue, ocular fluids, spinal fluid, a throat swab, breath, hair, finger nails, skin, biopsy, placental fluid, amniotic fluid, cord blood, lymphatic fluids, cavity fluids, sputum, pus, microbiota, meconium, breast milk and/or other excretions. The samples may include nasopharyngeal wash. Nasal swabs, throat swabs, stool samples, hair, finger nail, ear wax, breath, and other solid, semi-solid, or gaseous samples may be processed in an extraction buffer, e.g., for a fixed or variable amount of time, prior to their analysis. The extraction buffer or an aliquot thereof may then be processed similarly to other fluid samples if desired. Examples of tissue samples of the subject may include but are not limited to, connective tissue, muscle tissue, nervous tissue, epithelial tissue, cartilage, cancerous sample, or bone.

In certain embodiments, the subject may be a human or a non-human animal. The subject may be a mammal, vertebrate, such as murines, simians, humans, farm animals, sport animals, or pets. In some embodiments, the subject may be a patient. In other embodiments, the subject may be diagnosed with a disease, or the subject may not be diagnosed with a disease. In some embodiments, the subject may be a healthy subject.

### Device

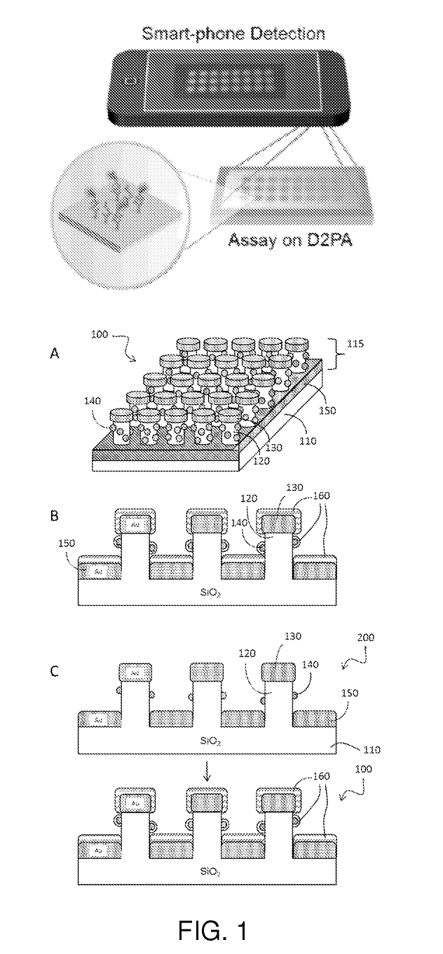

As summarized above, aspects of the method include processing the signal enhancing detector output with a device configured to acquire the detector output as input data and process the input data to generate a report. Any device suitable for acquiring the detector output as input data and processing the input data to generate a report may be used. In some embodiments, the device includes an optical recording apparatus that is configured to acquire an optical detector output as input data (FIG. 1). In certain instances, the optical recording apparatus is a camera, such as a digital camera. The term “digital camera” denotes any camera that includes as its main component an image-taking apparatus provided with an image-taking lens system for forming an optical image, an image sensor for converting the optical image into an electrical signal, and other components, examples of such cameras including digital still cameras, digital movie cameras, and Web cameras (i.e., cameras that are connected, either publicly or privately, to an apparatus connected to a network to permit exchange of images, including both those connected directly to a network and those connected to a network by way of an apparatus, such as a personal computer, having an information processing capability). In one example, the input data may include video imaging that may capture changes over time. For example, a video may be acquired to provide evaluation on dynamic changes in the sample.

With reference to FIG. 1, a D2PA array 100 may comprise: (a) substrate 110; and (b) one or a plurality of pillars 115 extending from a surface of the substrate, wherein at least one of the pillars comprises a pillar body 120, metallic disc 130 on top of the pillar, metallic back plane 150 at the foot of the pillar, the metallic back plane covering a substantial portion of the substrate surface near the foot of the pillar; metallic dot structure 130 disposed on sidewall of the pillar and molecular adhesion layer 160 that covers at least a part of the metallic dot structure, and/or the metal disc, and/or the metallic back plane. The underlying structure in this device has been referred as “disk-coupled dots-on-pillar antenna array, (D2PA)” and examples are them have been described (see, e.g., Li et al Optics Express 2011 19, 3925-3936 and WO2012/024006, which are incorporated by reference).

The exterior surface of molecular adhesion layer 160 may comprise a capture-agent-reactive group, i.e., a reactive group that can chemically react with capture agents, e.g., an amine-reactive group, a thiol-reactive group, a hydroxyl-reactive group, an imidazolyl-reactive group and a guanidinyl-reactive group. For illustrative purposes, the molecular adhesion layer 160 covers all of the exposed surface of metallic dot structure 160, metal disc 130, and metallic back plane 150. However, for practical purposes, adhesion layer 160 need only part of the exposed surface of metallic dot structure 160, metal disc 130, or metallic back plane 150. As shown, in certain cases, substrate 110 may be made of a dielectric (e.g., SiO₂) although other materials may be used, e.g., silicon, GaAs, polydimethylsiloxane (PDMS), poly(methyl methacrylate) (PMMA). Likewise, the metal may be gold, silver, platinum, palladium, lead, iron, titanium, nickel, copper, aluminum, alloy thereof, or combinations thereof, although other materials may be used, as long as the materials' plasma frequency is higher than that of the light signal and the light that is used to generate the light signal.

Device 100 may be characterized in that it amplifies a light signal that is proximal to the exterior surface of the adhesion layer.

In some embodiments, the dimensions of one or more of the parts of the pillars or a distance between two components may be that is less than the wavelength of the amplified light. For example, the lateral dimension of the pillar body 120, the height of pillar body 120, the dimensions of metal disc 130, the distances between any gaps between metallic dot structures 140, the distances between metallic dot structure 140 and metallic disc 130 may be smaller than the wavelength of the amplified light. As illustrated in FIG. 1, the pillars may be arranged on the substrate in the form of an array. In particular cases, the nearest pillars of the array may be spaced by a distance that is less than the wavelength of the light. The pillar array can be periodic and aperiodic.

The device may be disposed within a container, e.g., a well of a multi-well plate. The device also can be the bottom or the wall of a well of a multi-well plate. The devices may be disposed inside a microfluidic channel (channel width of 1 to 1000 micrometers) or nanofluidic channel (channel width less 1 micrometer) or a part of inside wall of such channels.

A subject nanodevice 100 may be fabricated by coating a so-called “disc-coupled dots-an-pillar antenna array” 200 (i.e., a “D2PA”, which is essentially composed of substrate 110 and a plurality of pillars that comprise pillar body 120, metallic disc 130, metallic back plane 150 and metallic dot structures 140 with a molecular adhesion layer 160. A detailed description an exemplary D2PA that can be employed in a subject nanodevice are provided in WO2012/024006, which is incorporated by reference herein for disclosure for all purposes.

In certain embodiments, the optical recording apparatus has a sensitivity that is lower than the sensitivity of a high-sensitivity optical recording apparatus used in research/clinical laboratory settings. In certain cases, the optical recording apparatus used in the subject method has a sensitivity that is lower by 10 times or more, such as 100 times or more, including 200 times or more, 500 times or more, or 1,000 times or more than the sensitivity of a high-sensitivity optical recording apparatus used in research/clinical laboratory settings.

In certain embodiments, the device acquires the detector output by means of an adaptor that forms an interface between the device and the detector. In certain embodiments, the interface is universal to be compatible with any device suitable for performing the subject method. Interfaces of interest include, but are not limited to, USB, firewire, Ethernet, etc. In certain embodiments, the device acquires the detector output by wireless communication, including cellular, Bluetooth, WiFi, etc.

In certain embodiments, the device may have a video display. Video displays may include components upon which a display page may be displayed in a manner perceptible to a user, such as, for example, a computer monitor, cathode ray tube, liquid crystal display, light emitting diode display, touchpad or touchscreen display, and/or other means known in the art for emitting a visually perceptible output. In certain embodiments, the device is equipped with a touch screen for displaying information, such as the input data acquired from the detector and/or the report generated from the processed data, and allowing information to be entered by the subject.

In certain embodiments, the device is equipped with vibration capabilities as a way to alert the subject, for example, of a report generated upon processing the detector output or in preparation for acquiring an output from the detector.

In certain embodiments, the subject device is configured to process the input data acquired from the signal enhancing detector. The device may be configured in any suitable way to process the data for use in the subject methods. In certain embodiments, the device has a memory location to store the data and/or store instructions for processing the data and/or store a database. The data may be stored in memory in any suitable format.

In certain embodiments, the device has a processor to process the data. In certain embodiments, the instructions for processing the data may be stored in the processor, or may be stored in a separate memory location. In some embodiments, the device may contain a software to implement the processing.

In certain embodiments, a device configured to process input data acquired from the detector contains software implemented methods to perform the processing. Software implemented methods may include one or more of: image acquisition algorithms; image processing algorithms; user interface methods that facilitate interaction between user and computational device and serves as means for data collection, transmission and analysis, communication protocols; and data processing algorithms. In certain embodiments, image processing algorithms include one or more of: a particle count, a LUT (look up table) filter, a particle filter, a pattern recognition, a morphological determination, a histogram, a line profile, a topographical representation, a binary conversion, or a color matching profile.

In certain embodiments, the device is configured to display information on a video display or touchscreen display when a display page is interpreted by software residing in memory of the device. The display pages described herein may be created using any suitable software language such as, for example, the hypertext mark up language (“HTML”), the dynamic hypertext mark up language (“DHTML”), the extensible hypertext mark up language (“XHTML”), the extensible mark up language (“XML”), or another software language that may be used to create a computer file displayable on a video or other display in a manner perceivable by a user. Any computer readable media with logic, code, data, instructions, may be used to implement any software or steps or methodology. Where a network comprises the Internet, a display page may comprise a webpage of a suitable type.

A display page according to the invention may include embedded functions comprising software programs stored on a memory device, such as, for example, VBScript routines, JScript routines, JavaScript routines, Java applets, ActiveX components, ASP.NET, AJAX, Flash applets, Silverlight applets, or AIR routines.

A display page may comprise well known features of graphical user interface technology, such as, for example, frames, windows, scroll bars, buttons, icons, and hyperlinks, and well known features such as a “point and click” interface or a touchscreen interface. Pointing to and clicking on a graphical user interface button, icon, menu option, or hyperlink also is known as “selecting” the button, option, or hyperlink A display page according to the invention also may incorporate multimedia features, multi-touch, pixel sense, IR LED based surfaces, vision-based interactions with or without cameras.

A user interface may be displayed on a video display and/or display page. The user interface may display a report generated based on analyzed data relating to the sample, as described further below.

The processor may be configured to process the data in any suitable way for use in the subject methods. The data is processed, for example, into binned data, transformed data (e.g., time domain data transformed by Fourier Transform to frequency domain), or may be combined with other data. The processing may put the data into a desired form, and may involve modifying the format of data. Processing may include detection of a signal from a sample, correcting raw data based on mathematical manipulation or correction and/or calibrations specific for the device or reagents used to examine the sample; calculation of a value, e.g., a concentration value, comparison (e.g., with a baseline, threshold, standard curve, historical data, or data from other sensors), a determination of whether or not a test is accurate, highlighting values or results that are outliers or may be a cause for concern (e.g., above or below a normal or acceptable range, or indicative of an abnormal condition), or combinations of results which, together, may indicate the presence of an abnormal condition, curve-fitting, use of data as the basis of mathematical or other analytical reasoning (including deductive, inductive, Bayesian, or other reasoning), and other suitable forms of processing. In certain embodiments, processing may involve comparing the processed data with a database stored in the device to retrieve instructions for a course of action to be performed by the subject.

In certain embodiments, the device may be configured to process the input data by comparing the input data with a database stored in a memory to retrieve instructions for a course of action to be performed by the subject. In some embodiments, the database may contain stored information that includes a threshold value for the analyte of interest. The threshold value may be useful for determining the presence or concentration of the one or more analyte. The threshold value may be useful for detecting situations where an alert may be useful. The data storage unit may include records or other information that may be useful for generating a report relating to the sample.

In certain embodiments, the device may be configured to acquire data that is not an output from the signal enhancing detector. Thus in certain cases, the device may be configured to acquire data that is not representative of the sample provided by the subject but may still be representative of the subject. Such data include, but are not limited to the age, sex, height, weight, individual and family medical history, etc. In certain embodiments, the device is configured to process the input data acquired from the detector output combined with data that was acquired independently of the detector output.

In certain embodiments the device may be configured to communicate over a network such as a local area network (LAN), wide area network (WAN) such as the Internet, personal area network, a telecommunications network such as a telephone network, cell phone network, mobile network, a wireless network, a data-providing network, or any other type of network. In certain embodiments the device may be configured to utilize wireless technology, such as Bluetooth or RTM technology. In some embodiments, the device may be configured to utilize various communication methods, such as a dial-up wired connection with a modem, a direct link such as TI, ISDN, or cable line. In some embodiments, a wireless connection may be using exemplary wireless networks such as cellular, satellite, or pager networks, GPRS, or a local data transport system such as Ethernet or token ring over a LAN. In some embodiments, the device may communicate wirelessly using infrared communication components.

In certain embodiments, the device is configured to receive a computer file, which can be stored in memory, transmitted from a server over a network. The device may receive tangible computer readable media, which may contain instructions, logic, data, or code that may be stored in persistent or temporary memory of the device, or may somehow affect or initiate action by the device. One or more devices may communicate computer files or links that may provide access to other computer files.

In some embodiments, the device is a personal computer, server, laptop computer, mobile device, tablet, mobile phone, cell phone, satellite phone, smartphone (e.g., iPhone, Android, Blackberry, Palm, Symbian, Windows), personal digital assistant, Bluetooth device, pager, land-line phone, or other network device. Such devices may be communication-enabled devices. The term “mobile phone” as used herein refers to a telephone handset that can operate on a cellular network, a Voice-Over IP (VoIP) network such as Session Initiated Protocol (SIP), or a Wireless Local Area Network (WLAN) using an 802.11x protocol, or any combination thereof. In certain embodiments, the device can be hand-held and compact so that it can fit into a consumer's wallet and/or pocket (e.g., pocket-sized).

In certain embodiments, the method includes transmitting the sample-derived data to a remote location where the transmitted data is analyzed. The remote location may be a location that is different from the location where the device is located. The remote location may include, but is not limited to, a hospital, doctor's office or other medical facility, or a research laboratory. In some instances, the remote location may have a computer, e.g., a server, that is configured to communicate with (i.e. receive information from and transmit information to) the device over a network. In some embodiments, the device may transmit data to a cloud computing infrastructure. The device may access the cloud computing infrastructure. In some embodiments, on-demand provision of computational resources (data, software) may occur via a computer network, rather than from a local computer. The device may contain very little software or data (perhaps a minimal operating system and web browser only), serving as a basic display terminal connected to the Internet. Since the cloud may be the underlying delivery mechanism, cloud-based applications and services may support any type of software application or service. Information provided by the device and/or accessed by the devices may be distributed over various computational resources. Alternatively, information may be stored in one or more fixed data storage unit or database.

In certain embodiments, the remote location includes a central database stored in a data storage unit that receives and analyzes the data transmitted from the device. The data storage units may be capable of storing computer readable media which may include code, logic, or instructions for the processor to perform one or more step. In some embodiments, the received data is analyzed in a comparative fashion with data contained in the central database and the result sent back to the subject. Analyzing may include correcting raw data based on mathematical manipulation or correction and/or calibrations specific for the device or reagents used to examine the sample; calculation of a value, e.g., a concentration value, comparison (e.g., with a baseline, threshold, standard curve, historical data, or data from other sensors), a determination of whether or not a test is accurate, highlighting values or results that are outliers or may be a cause for concern (e.g., above or below a normal or acceptable range, or indicative of an abnormal condition), or combinations of results which, together, may indicate the presence of an abnormal condition, curve-fitting, use of data as the basis of mathematical or other analytical reasoning (including deductive, inductive, Bayesian, or other reasoning), and other suitable forms of processing.

In certain embodiments, analyzing may involve comparing the analyzed data with a database stored in a data storage unit at the remote location to retrieve instructions for a course of action to be performed by the subject. In some embodiments, the database may contain stored information that includes a threshold value for the analyte of interest. The threshold value may be useful for determining the presence or concentration of the one or more analyte. The threshold value may be useful for detecting situations where an alert may be useful. The data storage unit may include any other information relating to sample preparation or clinical tests that may be run on a sample. The data storage unit may include records or other information that may be useful for generating a report relating to the analyzed data.

In certain embodiments, a health care professional is at the remote location. In other embodiments, a health care professional has access to the data transmitted by the device at a third location that is different from the remote location or the location of the device. A health care professional may include a person or entity that is associated with the health care system. A health care professional may be a medical health care provider. A health care professional may be a doctor. A health care professional may be an individual or an institution that provides preventive, curative, promotional or rehabilitative health care services in a systematic way to individuals, families and/or communities. Examples of health care professionals may include physicians (including general practitioners and specialists), dentists, physician assistants, nurses, midwives, pharmaconomists/pharmacists, dietitians, therapists, psychologists, chiropractors, clinical officers, physical therapists, phlebotomists, occupational therapists, optometrists, emergency medical technicians, paramedics, medical laboratory technicians, medical prosthetic technicians, radiographers, social workers, and a wide variety of other human resources trained to provide some type of health care service. A health care professional may or may not be certified to write prescriptions. A health care professional may work in or be affiliated with hospitals, health care centers and other service delivery points, or also in academic training, research and administration. Some health care professionals may provide care and treatment services for patients in private homes. Community health workers may work outside of formal health care institutions. Managers of health care services, medical records and health information technicians and other support workers may also be health care professionals or affiliated with a health care provider.

In some embodiments, the health care professional may already be familiar with the subject or have communicated with the subject. The subject may be a patient of the health care professional. In some instances, the health care professional may have prescribed the subject to undergo a clinical test. In one example, the health care professional may be the subject's primary care physician. The health care professional may be any type of physician for the subject (including general practitioners, and specialists).

Thus, a health care professional may analyze or review the data transmitted from the device and/or the results of an analysis performed at a remote location. In certain embodiments, the health care professional may send to the subject instructions or recommendations based on the data transmitted by the device and/or analyzed at the remote location.

### Report

An aspect of certain embodiments includes a device configured to generate a report upon processing the input data acquired from the detector output. In some embodiments, the report may contain any suitable information that is pertinent to the health status of the subject represented by the sample provided by the subject. The report may include: light data, including light intensity, wavelength, polarization, and other data regarding light, e.g., output from optical detectors such as photomultiplier tubes, photodiodes, charge-coupled devices, luminometers, spectrophotometers, cameras, and other light sensing components and devices, including absorbance data, transmittance data, turbidity data, luminosity data, wavelength data (including intensity at one, two, or more wavelengths or across a range of wavelengths), reflectance data, refractance data, birefringence data, polarization, and other light data; image data, e.g., data from digital cameras; the identifier information associated with the signal enhancing detector used to acquire the data; the processed data, as described above, etc. The report may represent qualitative or quantitative aspects of the sample.

In certain aspects, the report may indicate to the subject the presence or absence of an analyte, the concentration of an analyte, the presence or absence of a condition, the probability or likelihood that the subject has a condition, the likelihood of developing a condition, the change in likelihood of developing a condition, the progression of a condition, etc. The condition reported may include, but not limited to: cancer; inflammatory disease, such as arthritis; metabolic disease, such as diabetes; ischemic disease, such as stroke or heart attack; neurodegenerative disease, such as Alzheimer's Disease or Parkinson's Disease; organ failure, such as kidney or liver failure; drug overdose; stress; fatigue; muscle damage; pregnancy-related conditions, such as non-invasive prenatal testing, etc. In certain embodiments, the report contains instructions urging or recommending the patient to take action, such as seek medical help, take medication, stop an activity, start an activity, etc. The report may include an alert. One example of an alert may be if an error is detected on the device, or if an analyte concentration exceeds a predetermined threshold. The content of the report may be represented in any suitable form, including text, graphs, graphics, animation, color, sound, voice, and vibration.

In certain embodiment, the report provides an action advice to the subject who uses the subject device, e.g., a mobile phone. The advices will be given according to the test data by the devices (e.g. detectors plus mobile phone) together with one or several data sets, including but not limited to, the date preloaded on the mobile devices, data on a storage device that can accessed, where the storage device can be locally available or remotely accessible.

The advices include, but not limited to, one of the following: (i) normal (have a good day), (ii) should be monitored frequently; (iii) the following parameters should be checked closely (and list the parameters), (iv) should check every day, because subject's specific parameters on the boarder lines, (v) should visit doctor within certain days, because specific parameters are mild above to the threshold; (vi) should see doctor immediately, and (vii) should go to an emergency room immediately.

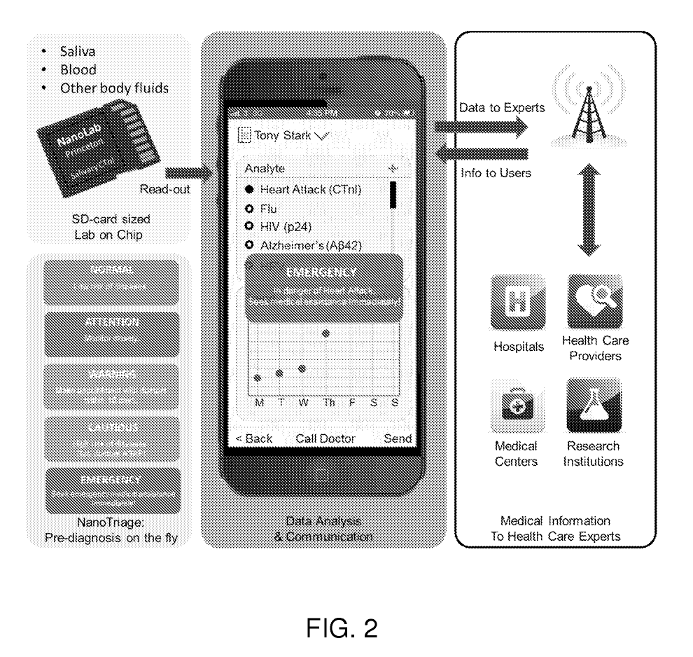

In certain embodiments, each of the advices above has its own color in scheme in the mobile phone displays. One example is given in FIG. 2.

In some embodiments, when the device concludes that a subject needs to see a physician or go an emergency room, the device automatically sends such request to a physician and an emergency room.

In some embodiments, when the automatically send request by the devices are not responded by a physician or an emergency room, the device will repeatedly send the request in certain time interval.

In certain embodiments, the report may provide a warning for any conflicts that may arise between an advice based on information derived from a sample provided by a subject and any contraindications based on a health history or profile of the subject.

### Method of Monitoring the Health of a Subject

In performing the subject method, the sample provided from a subject may be applied to the signal enhancing detector by any suitable method, including contacting the sample with a sample-receiving area of a signal enhancing detector, e.g., using a pipet, dropper, syringe, etc. In certain embodiments, when the signal enhancing detector is located on a support in a dipstick format, as described below, the sample may be applied to the signal enhancing detector by dipping a sample-receiving area of the dipstick into the sample.

Any volume of sample may be provided from the subject. Examples of volumes may include, but are not limited to, about 10 mL or less, 5 mL or less, 3 mL or less, 1 microliter (μL, also “uL” herein) or less, 500 μL, or less, 300 μL, or less, 250 μL, or less, 200 μL, or less, 170 μL, or less, 150 μL, or less, 125 μL, or less, 100 μL, or less, 75 μL, or less, 50 μL, or less, 25 μL, or less, 20 μL, or less, 15 μL, or less, 10 μL, or less, 5 μL, or less, 3 μL, or less, 1 μL, or less. The amount of sample may be about a drop of a sample. The amount of sample may be the amount collected from a pricked finger or fingerstick. The amount of sample may be the amount collected from a microneedle or a venous draw. Any volume, including those described herein, may be applied to the signal enhancing detector.

One or more, two or more, three or more, four or more, five or more, six or more, seven or more, eight or more, ten or more, twelve or more, fifteen or more, or twenty or more different types of samples may be provided from a subject. A single type of sample or a plurality of types of samples may be provided from the subject simultaneously or at different times. A single type of sample or a plurality of types of samples may be provided from the subject simultaneously or at different times.

A sample from the subject may be collected at one time, or at a plurality of times. The data may be collected at discrete points in time, or may be continuously collected over time. Data collected over time may be aggregated and/or processed. In some instances, data may be aggregated and may be useful for longitudinal analysis over time to facilitate screening, diagnosis, treatment, and/or disease prevention.

In certain instances, the period of time from applying the sample to the signal enhancing detector to generating an output that can be received by the device may range from 1 second to 30 minutes, such as 10 seconds to 20 minutes, 30 seconds to 10 minutes, including 1 minute to 5 minutes. In some instances, the period of time from applying the sample to the signal enhancing detector to generating an output that can be received by the device may be 1 hour or less, 30 minutes or less, 15 minutes or less, 10 minutes or less, 5 minutes or less, 3 minutes or less, 1 minute or less, 50 seconds or less, 40 seconds or less, 30 seconds or less, 20 seconds or less, 10 seconds or less, 5 seconds or less, 2 seconds or less, 1 second or less, or even shorter. In some instances, the period of time from applying the sample to the signal enhancing detector to generating an output that can be received by the device may be 100 milliseconds or more, including 200 milliseconds or more, such as 500 milliseconds or more, 1 second or more, 10 seconds or more, 30 seconds or more, 1 minute or more, 5 minutes or more, or longer.

In certain embodiments, the subject method includes processing the detector output with a device to generate a report. The detector output may be processed by the device to generate a report by any suitable method, as described above.

Embodiments of the method may further include receiving a report generated by the device. The report may be received in any convenient form, including, but not limited to, by viewing the report displayed on a screen on the device, by viewing an electronic mail or text message sent to the subject, by listening to an audio message generated by the device, by sensing a vibration generated by the device, etc.

Transmitting the data to a remote location may be achieved by any convenient method, as described above. Such transmissions may be via electronic signals, radiofrequency signals, optical signals, cellular signals, or any other type of signals that may be transmitted via a wired or wireless connection. Any transmission of data or description of electronic data or transmission described elsewhere herein may occur via electronic signals, radiofrequency signals, optical signals, cellular signals, or any other type of signals that may be transmitted via a wired or wireless connection. The transmitted data may include the input data and/or the processed data and/or the generated report. The transmitted data may also include data that was not acquired from the signal enhancing detector, i.e., data that does not represent an aspect of the sample obtained from the subject, but does represent other aspects of the subject, as described above.

In certain embodiments, the method includes receiving the analyzed data. The analyzed data may be received by the subject using any convenient method, including, but not limited to, by viewing the analyzed data displayed on a screen on the device, by viewing an electronic mail or text message sent to the subject, by listening to an audio message generated by the device, by sensing a vibration generated by the device, etc.

### Systems

As summarized above, aspects of the invention include systems that find use in practicing the subject method. In some embodiments the system includes a device configured to: receive as input data an output from a signal enhancing detector; process the input data to generate a report; and receive the report, wherein the signal enhancing detector is configured to indicate the output by obtaining a sample provided from the subject and generating an output that is representative of the sample (FIG. 2).

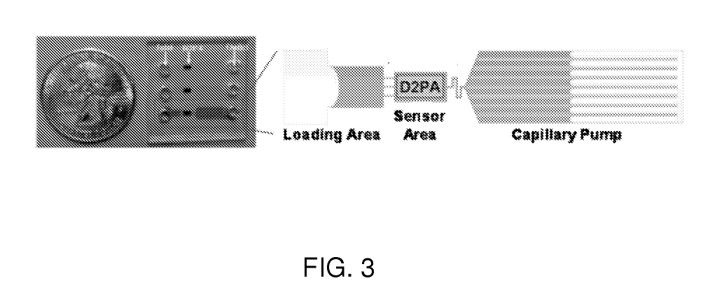

In certain embodiments, the signal enhancing detector is on a dipstick structure or a lateral flow format, examples of which are described in, e.g., U.S. Pat. No. 6,660,534, incorporated herein by reference. In other embodiments, the signal enhancing detector is a microfluidic device (FIG. 3). A “microfluidic device” is a device that is configured to control and manipulate fluids geometrically constrained to a small scale (e.g., sub-millimeter). Embodiments of the microfluidic devices include a detection region configured to receive a sample and indicate an output that is representative of the sample. In some embodiments, the signal enhancing detector is a lab-on-a-chip apparatus.

In certain embodiments, the signal enhancing detector includes a nanosensor. In certain embodiment, the signal enhancing detector includes a disk-coupled dots-on-pillar antenna array (D2PA). Exemplary signal enhancing detectors that find use in the present method are disclosed in, e.g., U.S. Pub. Nos. 2014/0154668 and 2014/0045209, which are hereby incorporated by reference.

The terms “disk-coupled dots-on-pillar antenna array” and “D2PA” as used herein refer to the device illustrated in FIGS. 1, 3 and 4, where the array comprises: (a) substrate; and (b) a D2PA structure, on the surface of the substrate, comprising one or a plurality of pillars extending from a surface of the substrate, wherein at least one of the pillars comprises a pillar body, metallic disc on top of the pillar, metallic back plane at the foot of the pillar, the metallic back plane covering a substantial portion of the substrate surface near the foot of the pillar; metallic dot structure disposed on sidewall of the pillar. The D2PA amplifies a light signal that is proximal to the surface of the D2PA. The D2PA enhances local electric field and local electric field gradient in regions that is proximal to the surface of the D2PA. The light signal includes light scattering, light diffraction, light absorption, nonlinear light generation and absorption, Raman scattering, chromaticity, luminescence that includes fluorescence, electroluminescence, chemiluminescence, and electrochemiluminescence.

A D2PA array may also comprise a molecular adhesion layer that covers at least a part of said metallic dot structure, said metal disc, and/or said metallic back plane and, optionally, a capture agent that specifically binds to an analyte, wherein said capture agent is linked to the molecular adhesion layer of the D2PA array. The nanosensor can amplify a light signal from an analyte, when said analyte is bound to the capture agent. One preferred SAL embodiment is that the dimension of one, several or all critical metallic and dielectric components of SAL are less than the wavelength of the light in sensing. Details of the physical structure of disk-coupled dots-on-pillar antenna arrays, methods for their fabrication, methods for linking capture agents to disk-coupled dots-on-pillar antenna arrays and methods of using disk-coupled dots-on-pillar antenna arrays to detect analytes are described in a variety of publications including WO2012024006, WO2013154770, Li et al (Optics Express 2011 19, 3925-3936), Zhang et al (Nanotechnology 2012 23: 225-301); and Zhou et al (Anal. Chem. 2012 84: 4489) which are incorporated by reference for those disclosures.

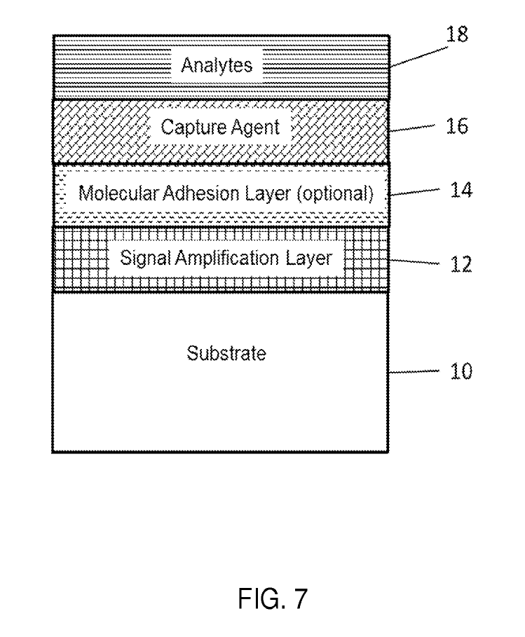

As illustrated by the box diagram in FIG. 7, a nanosensor for sensing an analyte 18, comprise: (a) a substrate 10; (b) a signal amplification layer (SAL) 12 on top of the substrate 10, (c) an optional molecular adhesion layer 14 on the surface of the SAL 12, (d) a capture agent 16 that specifically binds to the analyte 18, wherein the nanosensor amplifies a light signal from an analyte 18, when the analyte is bound to the capture agent 16. The SAL, comprising metallic and non-metallic micro/nanostructures, amplifies the sensing signal of the analytes captured by the capture agent, without an amplification of the number of molecules. Furthermore, such amplification is most effect within the very small depth (˜100 nm) from the SAL surface.

In certain embodiments, the analytes are labeled with a light-emitting label, either prior to or after it is bound to the capture agent. The analytes are also termed as biomarkers in certain embodiments.

In some embodiments, the signal enhancing detector includes liquid handling components, such as microfluidic fluid handling components (FIG. 3). The fluid handling components may be configured to direct one or more fluids through the signal enhancing detector. In some instances, the fluid handling components are configured to direct fluids, such as, but not limited to, a sample solution, buffers and the like. Liquid handling components may include, but are not limited to, passive pumps and microfluidic channels. In some cases, the passive pumps are configured for capillary action-driven microfluidic handling and routing of fluids through the signal enhancing detectors disclosed herein. In certain instances, the microfluidic fluid handling components are configured to deliver small volumes of fluid, such as 1 mL or less, such as 500 μL or less, including 100 μL or less, for example 50 μL or less, or 25 μL or less, or 10 μL or less, or 5 μL or less, or 1 μL or less. Thus, in certain embodiments, no external source of power is required to operate the system.

In certain embodiments, the signal enhancing detector has dimensions in the range of 5 mm×5 mm to 100 mm×100 mm, including dimensions of 50 mm×50 mm or less, for instance 25 mm×25 mm or less, or 10 mm×10 mm or less. In certain embodiments, the signal enhancing detector has a thickness in the range of 5 mm to 0.1 mm, such as 3 mm to 0.2 mm, including 2 mm to 0.3 mm, or 1 mm to 0.4 mm.

In some embodiments, the signal enhancing detector may have an identifier. An identifier may be a physical object formed on the signal enhancing detector. For example, the identifier may be read by a device of the subject system. Thus, in some instances, the output from a signal enhancing detector may include an identifier. In some embodiments, a camera may capture an image of the identifier and the image may be analyzed to identify the signal enhancing detector. In one example, the identifier may be a barcode. A barcode may be a 1D or 2D barcode. In some embodiments, the identifier may emit one or more signal that may identify the signal enhancing detector. For example, the identifier may provide an infrared, ultrasonic, optical, audio, electrical, or other signal that may indicate the identity of the signal enhancing detector. The identifier may utilize a radiofrequency identification (RFID) tag. The identifier may be stored on a memory of the signal enhancing detector. In one example, the identifier may be a computer readable medium.

The identifier may contain information that allows a device configured to acquire the output from a signal enhancing detector and process the output to determine the specific type of signal enhancing detector used to produce an output that is representative of a sample. In certain embodiments, the identifier provides a key to a database that associates each identifier key to information specific to the type of signal enhancing detector used to produce an output that is representative of a sample. The information specific to the type of signal enhancing detector may include, but are not limited to, the identity of the analytes which the signal enhancing detector is configured to bind, the coordinates of the position where a specific analyte may bind on the signal enhancing detector, the sensitivity of detection for each analyte, etc. The database may contain other information relevant to a specific signal enhancing detector, including an expiration date, lot number, etc. The database may be present on the device, provided on a computer-readable medium, or may be accessible by the device on a remote server.

In certain embodiments, the system has a sensitivity of detection that is higher than a system that does not have a physical signal amplification process but uses a high-sensitivity laboratory grade reader by 10 times or more, including 100 times or more, such as 200 times or more, 500 times or more, 1000 times or more, or higher. In certain embodiments, the system has a sensitivity of detection that is higher than a system that does not have a physical signal amplification process but uses a high-sensitivity laboratory grade reader by 10 to 10,000 fold, e.g., 100 to 5000 fold, including 200 to 2000 fold, or 500 to 1000 fold.

Embodiments of the system include a device configured to generate a report upon processing the output from a signal enhancing detector and provide the report to the subject. In some embodiments, the report may include diagnostic information about the subject for a condition, such as a disease. In certain embodiments, the system achieves a diagnostic accuracy of 75% or more, such as 80% or more, including 85% or more, or 90% or more.

### Utility

The subject methods and systems find use in a variety of different applications where determination of the presence or absence, and/or quantification of one or more analytes in a sample and/or monitoring the health of an individual is desired. For example, the subject systems and methods find use in the detection of proteins, peptides, nucleic acids, and the like. In some cases, the subject systems and methods find use in the detection of proteins.

In certain embodiments, the subject systems and methods find use in the detection of nucleic acids, proteins, or other biomolecules in a sample. The methods may include the detection of a set of biomarkers, e.g., two or more distinct protein biomarkers, in a sample. For example, the methods may be used in the rapid, clinical detection of two or more disease biomarkers in a biological sample, e.g., as may be employed in the diagnosis of a disease condition in a subject, or in the ongoing management or treatment of a disease condition in a subject, etc. As described above, communication to a physician or other health-care provider may better ensure that the physician or other health-care provider is made aware of, and cognizant of, possible concerns and may thus be more likely to take appropriate action.

In certain embodiments, the subject systems and methods find use in detecting biomarkers. In some cases, the subject systems and methods may be used to detect the presence or absence of particular biomarkers, as well as an increase or decrease in the concentration of particular biomarkers in blood, plasma, serum, or other bodily fluids or excretions, such as but not limited to urine, blood, serum, plasma, saliva, semen, prostatic fluid, nipple aspirate fluid, lachrymal fluid, perspiration, feces, cheek swabs, cerebrospinal fluid, cell lysate samples, amniotic fluid, gastrointestinal fluid, biopsy tissue, and the like.

The presence or absence of a biomarker or significant changes in the concentration of a biomarker can be used to diagnose disease risk, presence of disease in an individual, or to tailor treatments for the disease in an individual. For example, the presence of a particular biomarker or panel of biomarkers may influence the choices of drug treatment or administration regimes given to an individual. In evaluating potential drug therapies, a biomarker may be used as a surrogate for a natural endpoint such as survival or irreversible morbidity. If a treatment alters the biomarker, which has a direct connection to improved health, the biomarker can serve as a surrogate endpoint for evaluating the clinical benefit of a particular treatment or administration regime. Thus, personalized diagnosis and treatment based on the particular biomarkers or panel of biomarkers detected in an individual are facilitated by the subject systems and methods. Furthermore, the early detection of biomarkers associated with diseases is facilitated by the high sensitivity of the subject devices and systems, as described above. Due to the capability of detecting multiple biomarkers with a mobile device, such as a smartphone, combined with sensitivity, scalability, and ease of use, the presently disclosed systems and methods find use in portable and point-of-care or near-patient molecular diagnostics.

In certain embodiments, the subject systems and methods find use in detecting biomarkers for a disease or disease state. In certain instances, the subject systems and methods find use in detecting biomarkers for the characterization of cell signaling pathways and intracellular communication for drug discovery and vaccine development. For example, the subject systems and methods may be used to detect and/or quantify the amount of biomarkers in diseased, healthy or benign samples. In certain embodiments, the subject systems and methods find use in detecting biomarkers for an infectious disease or disease state. In some cases, the biomarkers can be molecular biomarkers, such as but not limited to proteins, nucleic acids, carbohydrates, small molecules, and the like.

The subject systems and methods find use in diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

The subject systems and methods also find use in validation assays. For example, validation assays may be used to validate or confirm that a potential disease biomarker is a reliable indicator of the presence or absence of a disease across a variety of individuals. The short assay times for the subject systems and methods may facilitate an increase in the throughput for screening a plurality of samples in a minimum amount of time.

In some instances, the subject systems and methods can be used without requiring a laboratory setting for implementation. In comparison to the equivalent analytic research laboratory equipment, the subject devices and systems provide comparable analytic sensitivity in a portable, hand-held system. In some cases, the mass and operating cost are less than the typical stationary laboratory equipment. In addition, the subject systems and devices can be utilized in a home setting for over-the-counter home testing by a person without medical training to detect one or more analytes in samples. The subject systems and devices may also be utilized in a clinical setting, e.g., at the bedside, for rapid diagnosis or in a setting where stationary research laboratory equipment is not provided due to cost or other reasons.

### Kits

Aspects of the present invention include kits that provide a signal enhancing detector for monitoring the health of a subject and instructions for practicing the subject methods using a hand held device, e.g., a mobile phone. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Another means would be a computer readable medium, e.g., diskette, CD, DVD, Blu-Ray, computer-readable memory, etc., on which the information has been recorded or stored. Yet another means that may be present is a website address which may be used via the Internet to access the information at a removed site. The kit may further include a software for implementing a method for monitoring the health of a subject on a device, as described herein, provided on a computer readable medium. Any convenient means may be present in the kits.

### Samples, Health Conditions, and Applications

The samples from a subject, the health of a subject, and other applications of the present invention are further described below. Exemplary samples, health conditions, and application are also disclosed in, e.g., U.S. Pub. Nos. 2014/0154668 and 2014/0045209, which are hereby incorporated by reference.

The present inventions find use in a variety applications, where such applications are generally analyte detection applications in which the presence of a particular analyte in a given sample is detected at least qualitatively, if not quantitatively. Protocols for carrying out analyte detection assays are well known to those of skill in the art and need not be described in great detail here. Generally, the sample suspected of comprising an analyte of interest is contacted with the surface of a subject nanosensor under conditions sufficient for the analyte to bind to its respective capture agent that is tethered to the sensor. The capture agent has highly specific affinity for the targeted molecules of interest. This affinity can be antigen-antibody reaction where antibodies bind to specific epitope on the antigen, or a DNA/RNA or DNA/RNA hybridization reaction that is sequence-specific between two or more complementary strands of nucleic acids. Thus, if the analyte of interest is present in the sample, it likely binds to the sensor at the site of the capture agent and a complex is formed on the sensor surface. Namely, the captured analytes are immobilized at the sensor surface. After removing the unbounded analytes, the presence of this binding complex on the surface of the sensor (i.e. the immobilized analytes of interest) is then detected, e.g., using a labeled secondary capture agent.

Specific analyte detection applications of interest include hybridization assays in which the nucleic acid capture agents are employed and protein binding assays in which polypeptides, e.g., antibodies, are employed. In these assays, a sample is first prepared and following sample preparation, the sample is contacted with a subject nanosensor under specific binding conditions, whereby complexes are formed between target nucleic acids or polypeptides (or other molecules) that are complementary to capture agents attached to the sensor surface.

In one embodiment, the capture oligonucleotide is synthesized single strand DNA of 20-100 bases length, that is thiolated at one end. These molecules are immobilized on the nanodevices' surface to capture the targeted single-strand DNA (which may be at least 50 bp length) that has a sequence that is complementary to the immobilized capture DNA. After the hybridization reaction, a detection single strand DNA (which can be of 20-100 bp in length) whose sequence are complementary to the targeted DNA's unoccupied nucleic acid is added to hybridize with the target. The detection DNA has its one end conjugated to a fluorescence label, whose emission wavelength are within the plasmonic resonance of the nanodevice. Therefore by detecting the fluorescence emission emanate from the nanodevices' surface, the targeted single strand DNA can be accurately detected and quantified. The length for capture and detection DNA determine the melting temperature (nucleotide strands will separate above melting temperature), the extent of misparing (the longer the strand, the lower the misparing). One of the concerns of choosing the length for complementary binding depends on the needs to minimize misparing while keeping the melting temperature as high as possible. In addition, the total length of the hybridization length is determined in order to achieve optimum signal amplification.

A subject sensor may be employed in a method of diagnosing a disease or condition, comprising: (a) obtaining a liquid sample from a patient suspected of having the disease or condition, (b) contacting the sample with a subject nanosensor, wherein the capture agent of the nanosensor specifically binds to a biomarker for the disease and wherein the contacting is done under conditions suitable for specific binding of the biomarker with the capture agent; (c) removing any biomarker that is not bound to the capture agent; and (d) reading a light signal from biomarker that remain bound to the nanosensor, wherein a light signal indicates that the patient has the disease or condition, wherein the method further comprises labeling the biomarker with a light-emitting label, either prior to or after it is bound to the capture agent. As will be described in greater detail below, the patient may suspected of having cancer and the antibody binds to a cancer biomarker. In other embodiments, the patient is suspected of having a neurological disorder and the antibody binds to a biomarker for the neurological disorder.

The applications of the subject sensor include, but not limited to, (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

The detection can be carried out in various sample matrix, such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate.

In some embodiments, a subject biosensor can be used diagnose a pathogen infection by detecting a target nucleic acid from a pathogen in a sample. The target nucleic acid may be, for example, from a virus that is selected from the group comprising human immunodeficiency virus 1 and 2 (HIV-1 and HIV-2), human T-cell leukaemia virus and 2 (HTLV-1 and HTLV-2), respiratory syncytial virus (RSV), adenovirus, hepatitis B virus (HBV), hepatitis C virus (HCV), Epstein-Barr virus (EBV), human papillomavirus (HPV), varicella zoster virus (VZV), cytomegalovirus (CMV), herpes-simplex virus 1 and 2 (HSV-1 and HSV-2), human herpesvirus 8 (HHV-8, also known as Kaposi sarcoma herpesvirus) and flaviviruses, including yellow fever virus, dengue virus, Japanese encephalitis virus, West Nile virus and Ebola virus. The present invention is not, however, limited to the detection of nucleic acid, e.g., DNA or RNA, sequences from the aforementioned viruses, but can be applied without any problem to other pathogens important in veterinary and/or human medicine.

Human papillomaviruses (HPV) are further subdivided on the basis of their DNA sequence homology into more than 70 different types. These types cause different diseases. HPV types 1, 2, 3, 4, 7, 10 and 26-29 cause benign warts. HPV types 5, 8, 9, 12, 14, 15, 17 and 19-25 and 46-50 cause lesions in patients with a weakened immune system. Types 6, 11, 34, 39, 41-44 and 51-55 cause benign acuminate warts on the mucosae of the genital region and of the respiratory tract. HPV types 16 and 18 are of special medical interest, as they cause epithelial dysplasias of the genital mucosa and are associated with a high proportion of the invasive carcinomas of the cervix, vagina, vulva and anal canal. Integration of the DNA of the human papillomavirus is considered to be decisive in the carcinogenesis of cervical cancer. Human papillomaviruses can be detected for example from the DNA sequence of their capsid proteins L1 and L2. Accordingly, the method of the present invention is especially suitable for the detection of DNA sequences of HPV types 16 and/or 18 in tissue samples, for assessing the risk of development of carcinoma.

In some cases, the nanosensor may be employed to detect a biomarker that is present at a low concentration. For example, the nanosensor may be used to detect cancer antigens in a readily accessible bodily fluids (e.g., blood, saliva, urine, tears, etc.), to detect biomarkers for tissue-specific diseases in a readily accessible bodily fluid (e.g., a biomarkers for a neurological disorder (e.g., Alzheimer's antigens)), to detect infections (particularly detection of low titer latent viruses, e.g., HIV), to detect fetal antigens in maternal blood, and for detection of exogenous compounds (e.g., drugs or pollutants) in a subject's bloodstream, for example.

The following table provides a list of protein biomarkers that can be detected using the subject nanosensor (when used in conjunction with an appropriate monoclonal antibody), and their associated diseases. One potential source of the biomarker (e.g., “CSF”; cerebrospinal fluid) is also indicated in the table. In many cases, the subject biosensor can detect those biomarkers in a different bodily fluid to that indicated. For example, biomarkers that are found in CSF can be identified in urine, blood or saliva.

| Marker | disease |
|---|---|
| Aβ42, amyloid beta-protein (CSF) | Alzheimer's disease. |
| fetuin-A (CSF) | multiple sclerosis. |
| tau (CSF) | niemann-pick type C. |
| secretogranin II (CSF) | bipolar disorder. |
| prion protein (CSF) | Alzheimer disease, prion disease |
| Cytokines (CSF) | HIV-associated neurocognitive |
| | disorders |
| Alpha-synuclein (CSF) | parkinsonian disorders |
| | (neuordegenerative disorders) |
| tau protein (CSF) | parkinsonian disorders |
| neurofilament light chain (CSF) | axonal degeneration |
| parkin (CSF) | neuordegenerative disorders |
| PTEN induced putative kinase 1 (CSF) | neuordegenerative disorders |
| DJ-1 (CSF) | neuordegenerative disorders |
| leucine-rich repeat kinase 2 (CSF) | neuordegenerative disorders |
| mutated ATP13A2 (CSF) | Kufor-Rakeb disease |
| Apo H (CSF) | parkinson disease (PD) |
| ceruloplasmin (CSF) | PD |
| Peroxisome proliferator-activated | PD |
| receptor gamma coactivator- | |
| 1 alpha (PGC-1α)(CSF) | |
| transthyretin (CSF) | CSF rhinorrhea (nasal surgery |
| | samples) |
| Vitamin D-binding Protein (CSF) | Multiple Sclerosis Progression |
| proapoptotic kinase R (PKR) and its | AD |
| phosphorylated PKR (pPKR) (CSF) | |
| CXCL13 (CSF) | multiple sclerosis |
| IL-12p40, CXCL13 and IL-8 (CSF) | intrathecal inflammation |
| Dkk-3 (semen) | prostate cancer |
| p14 endocan fragment (blood) | Sepsis: Endocan, specifically |
| | secreted by activated-pulmonary |
| | vascular endothelial cells, is |
| | thought to play a key role in the |
| | control of the lung inflammatory |
| | reaction. |
| Serum (blood) | neuromyelitis optica |
| ACE2 (blood) | cardiovascular disease |
| autoantibody to CD25 (blood) | early diagnosis of esophageal |
| | squamous cell carcinoma |
| hTERT (blood) | lung cancer |
| CAI25 (MUC 16) (blood) | lung cancer |
| VEGF (blood) | lung cancer |
| sIL-2 (blood) | lung cancer |
| Osteopontin (blood) | lung cancer |
| Human epididymis protein 4 | ovarian cancer |
| (HE4) (blood) | |
| Alpha-Fetal Protein (blood) | pregnancy |
| Albumin (urine) | diabetics |
| albumin (urine) uria | albuminuria |
| microalbuminuria | kidney leaks |
| AFP (urine) | mirror fetal AFP levels |
| neutrophil gelatinase-associated | Acute kidney injury |
| lipocalin (NGAL) (urine) | |
| interleukin 18 (IL-18) (urine) | Acute kidney injury |
| Kidney Injury Molecule-1 | Acute kidney injury |
| (KIM-1) (urine) | |
| Liver Fatty Acid Binding Protein | Acute kidney injury |
| (L-FABP) (urine) | |
| LMP1 (saliva) | Epstein-Barr virus oncoprotein |
| | (nasopharyngeal carcinomas) |
| BARF1 (saliva) | Epstein-Barr virus oncoprotein |
| | (nasopharyngeal carcinomas) |
| IL-8 (saliva) | oral cancer biomarker |
| carcinoembryonic antigen | oral or salivary malignant tumors |
| (CEA) (saliva) | |
| BRAF, CCNI, EGRF, FGF19, | Lung cancer |
| FRS2, GREB1, and LZTS1 (saliva) | |
| alpha-amylase (saliva) | cardiovascular disease |
| carcinoembryonic antigen (saliva) | Malignant tumors of the oral cavity |
| CA 125 (saliva) | Ovarian cancer |
| IL8 (saliva) | spinalcellular carcinoma. |
| thioredoxin (saliva) | spinalcellular carcinoma. |
| beta-2 microglobulin levels-monitor | HIV |
| activity of the virus (saliva) | |
| tumor necrosis factor-alpha receptors- | HIV |
| monitor activity of the virus (saliva) | |
| CA15-3 (saliva) | breast cancer |

The health conditions that may be diagnosed or measured by the subject method, device and system include, but are not limited to: chemical balance; nutritional health; exercise; fatigue; sleep; stress; prediabetes; allergies; aging; exposure to environmental toxins, pesticides, herbicides, synthetic hormone analogs; pregnancy; menopause; and andropause.

As noted above, a subject nanosensor can be used to detect nucleic acid in a sample. A subject nanosensor may be employed in a variety of drug discovery and research applications in addition to the diagnostic applications described above. For example, a subject nanosensor may be employed in a variety of applications that include, but are not limited to, diagnosis or monitoring of a disease or condition (where the presence of an nucleic acid provides a biomarker for the disease or condition), discovery of drug targets (where, e.g., an nucleic acid is differentially expressed in a disease or condition and may be targeted for drug therapy), drug screening (where the effects of a drug are monitored by assessing the level of an nucleic acid), determining drug susceptibility (where drug susceptibility is associated with a particular profile of nucleic acids) and basic research (where is it desirable to identify the presence a nucleic acid in a sample, or, in certain embodiments, the relative levels of a particular nucleic acids in two or more samples).

In certain embodiments, relative levels of nucleic acids in two or more different nucleic acid samples may be obtained using the above methods, and compared. In these embodiments, the results obtained from the above-described methods are usually normalized to the total amount of nucleic acids in the sample (e.g., constitutive RNAs), and compared. This may be done by comparing ratios, or by any other means. In particular embodiments, the nucleic acid profiles of two or more different samples may be compared to identify nucleic acids that are associated with a particular disease or condition.

In some examples, the different samples may consist of an “experimental” sample, i.e., a sample of interest, and a “control” sample to which the experimental sample may be compared. In many embodiments, the different samples are pairs of cell types or fractions thereof, one cell type being a cell type of interest, e.g., an abnormal cell, and the other a control, e.g., normal, cell. If two fractions of cells are compared, the fractions are usually the same fraction from each of the two cells. In certain embodiments, however, two fractions of the same cell may be compared. Exemplary cell type pairs include, for example, cells isolated from a tissue biopsy (e.g., from a tissue having a disease such as colon, breast, prostate, lung, skin cancer, or infected with a pathogen etc.) and normal cells from the same tissue, usually from the same patient; cells grown in tissue culture that are immortal (e.g., cells with a proliferative mutation or an immortalizing transgene), infected with a pathogen, or treated (e.g., with environmental or chemical agents such as peptides, hormones, altered temperature, growth condition, physical stress, cellular transformation, etc.), and a normal cell (e.g., a cell that is otherwise identical to the experimental cell except that it is not immortal, infected, or treated, etc.); a cell isolated from a mammal with a cancer, a disease, a geriatric mammal, or a mammal exposed to a condition, and a cell from a mammal of the same species, preferably from the same family, that is healthy or young; and differentiated cells and non-differentiated cells from the same mammal (e.g., one cell being the progenitor of the other in a mammal, for example). In one embodiment, cells of different types, e.g., neuronal and non-neuronal cells, or cells of different status (e.g., before and after a stimulus on the cells) may be employed. In another embodiment of the invention, the experimental material is cells susceptible to infection by a pathogen such as a virus, e.g., human immunodeficiency virus (HIV), etc., and the control material is cells resistant to infection by the pathogen. In another embodiment of the invention, the sample pair is represented by undifferentiated cells, e.g., stem cells, and differentiated cells.

### Definitions

Most of the definitions used in the present invention are disclosed in, e.g., U.S. Pub. Nos. 2014/0154668 and 2014/0045209, which are hereby incorporated by reference. Furthermore, biomarkers are the analytes that are resulted to certain health conditions.

### EXEMPLARY EMBODIMENTS

Further examples are given below, they are include as a part of the present invention application and incorporated in its entirety.

### Example 1: Smart-Phone Based Personalized Medicine

With reference to FIG. 2, an exemplary method, according to an embodiment of the present disclosure, is shown below.
1. Having a disk-coupled dots-on-pillar antenna array (D2PA) chip
2. Put a droplet of sample (saliva, blood, sweet, urine, feats, . . . ) on the D2PA chip.
3. Reading the chip by smartphone
4. Smartphone displays: normal, attention, warning, caution, emergency, (see FIG. 2 for details)
5. Test info being transmitted to data base, physician, hospital, etc. (see FIG. 2 for details)
6. Instructions being transmitted back.
7. Person takes actions to do X.
8. The use of above test are: (a) daily health test, (b) disease/cancer monitoring, (c) patient off-hospital monitoring, (d) allergy, . . . .

### Example 2: Ultra-Sensitive, Rapid, Fluorescence Assay Platform for Disease/Cancer Early Diagnosis and Personalized Medicine

1. Overview. An assay platform, disk-coupled dots-on-pillar antenna array (D2PA)-Assay, that has demonstrated the detection of biomarkers (proteins or DNAs) with a sensitivity of 4-6 orders of magnitude higher than the existing best commercial technology has been developed. The developed assay platform can be broadly applied to sensitivity enhancement of nearly all fluorescence/luminescence based assays, and is fast, simple-to-use, and low cost. Already, it has demonstrated such sensitivity enhancement in detecting the biomarkers of Alzheimer's disease (AD), prostate cancers and breast cancer. The ultrasensitive assay platform also has enormous applications in other areas in human healthcare (allergy, food safety, etc) and other bio/chemical sensing areas (animal, agriculture, bio-threat detections, etc.)

2. Technology. Protein and DNA detection is universal and vital in biological study and medical diagnosis. Fluorescent assay (immuno or DNA), which identifies a targeted protein or DNA biomarker (i.e., analyte) by selectively tagging it with a detection agent (antibody or detecting DNA) labeled with fluorophores, is one of the most widely used and most sensitive methods. When excited by light, the fluorophore's fluorescent intensity is related to the existence and the concentration of the biomarker.

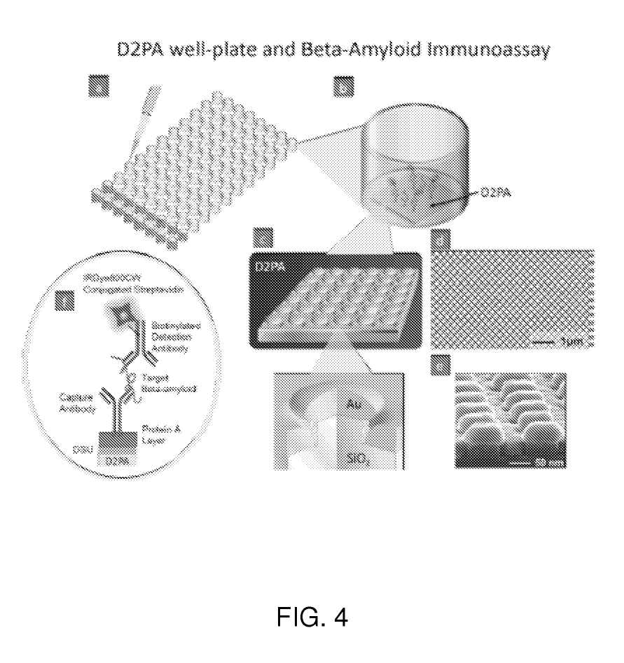

Fluorescence can be enhanced by metallic nanostructures through light focusing. The developed assay platform uses a special nanostructure surface, termed “disk-coupled dots-on-pillar antenna array” (D2PA), that couples subwavelength-size small metallic nanoparticles for focusing light with wavelength-size 3D antennas for good light absorption and radiation, drastically enhancing fluorescence for a given excitation power and hence fluorophore detection sensitivity (3 to 5 orders of magnitude). One example of the D2PA consists of a periodic dielectric pillar array (200 nm pitch and ˜100 nm diameter), a metallic disk (˜135 nm diameter) on top of each pillar, a metallic backplane on the foot of the pillars, subwavelength metallic nanodots randomly located on the pillar walls, and nanogaps between these metal components (FIG. 4). The metallic disk and the metallic back plane form a 3D cavity antenna.

FIG. 4. Immuno or DNA assay platform (D2PA assay) and Beta-amyloid (AR) Immunoassay.

(a) Schematic. D2PA assay plate at the bottom of a standard 96 well plate. (b) Zoomed-in. (c) Schematic, (d) top view and (e) close-up of scanning electron micrograph of the D2PA. And (f) Schematic of a fluorescent sandwich immunoassay placed on the bio-functioned D2PA plate (the coupling layer is DSU and Protein A)

Furthermore, technologies that can place the biomarkers at “hot-spots” (the highest enhancement locations), whereby these developed technologies further increase detection sensitivity by another 10 to 100 fold (so total 4 to 6 orders of magnitude), and technologies that can manufacture such structures uniformly, in large volume, and low cost, were developed.

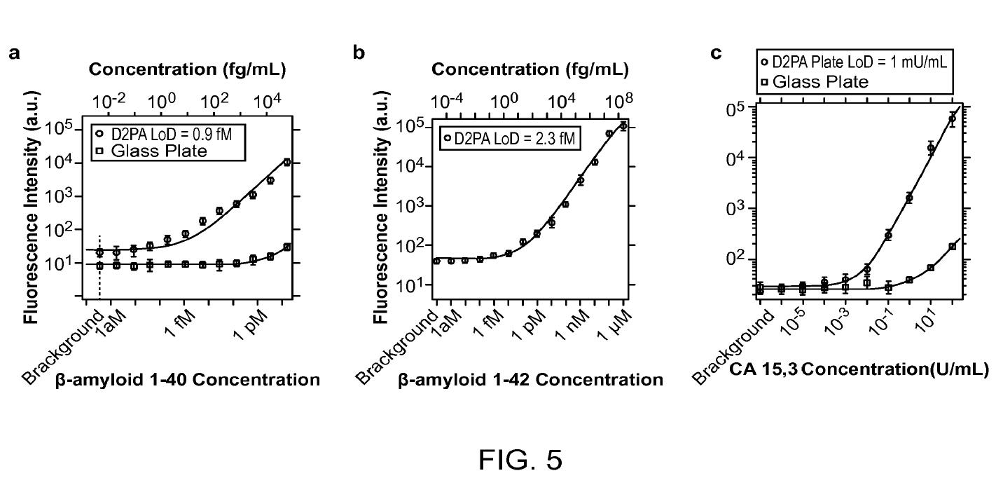

To form a biomarker assay, a coupling agent layer was coated on top of D2PA and then capture agent. After having captured the targeted biomarkers by the capture agent, labeled detection agent were used to selectively bond and identify the captured biomarker. For a given biomarker, a selective pair of capture and detection agents is used. Since the fluorescence enhancement in D2PA-Assay does not modify assay chemistry but only light radiation physics, such fluorescence enhancement can be broadly applied to all existing fluorescence assays. For example, in the detecting AD biomarker, Aβ-42/40, commercial “Aβ-42/40 ELISA kits” (Covance USA) were purchased, where the enzyme and the substrate were not used, but rather commercial streptavidinconjugated fluorescence (IRDye800CW) labels (Rockland USA) were attached to the detection agent. The rest of the kit was used as provided by the manufacturer. Similar assays on D2PA plate for detection of prostate specific antigen (PSA), and CA15.3 cancer and carcinoembryonic antigen (CEA) biomarkers were also implemented (FIG. 5).

FIG. 5 Immunoassay standard curves for different biomarkers on D2PA. (a) Measured fluorescence response of A640 standard on D2PA plate (circle) and glass plate (square). LoD=0.2 fM (D2PA) and 10 pM (glass), respectively (50,000 enhancement). (b) Aβ 42 LoD=2.3 fM with a broad dynamic range of 6 orders of magnitude. (c) CA15.3 LoD=0.001 U/mL for D2PA plate and 5 U/mL for glass plate. (5,000×).

An ultra-sensitive assay of the present disclosure allows (a) discovery of new biomarkers, (b) detection of a known biomarker in a different body fluid, where biomarker concentration much lower but sampling is much easier (noninvasively) (e.g. replace cerebrospinal fluid (CSF) sampling by saliva); and (c) diagnosis a test using smart phone rather than fancy ultra-high resolution reader.

3. Noninvasive early detection of Alzheimer's disease (AD). The concentrations of beta-amyloid (Aβ)-42 and tau in cerebrospinal fluid (CSF) are key biomarkers to diagnosis AD. However, the procedure for extracting CSF is very aggressive, requires specially trained professionals, has certain risks, and produce only a very small amount of CSF each time. Thus it would be advantageous to measure Aβ-42 concentration in saliva for AD diagnosis. The D2PA Aβ-42 assay has a LoD of 2.3 fg/mL (basic model) and 92 ag/mL (advanced model), which are ˜500 and 11,000 fold higher than previous methods.

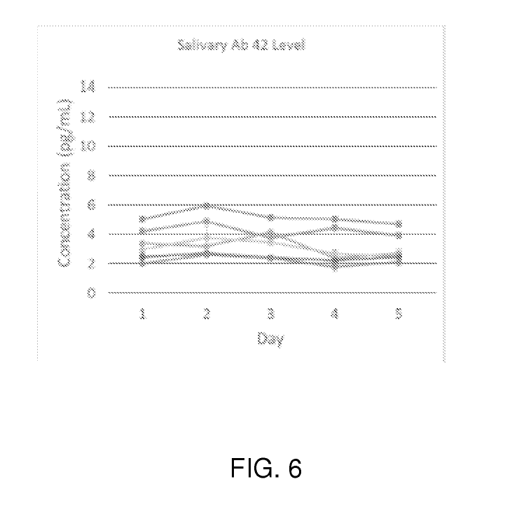

Using D2PA assay, the Aβ-42 concentration in saliva of 6 healthy males (all volunteers) in five consecutive days was measured (FIG. 6). The measured Aβ-42 concentrations were very consistent and stable in saliva, indicating the Aβ-42 in saliva is a good marker in AD study.

FIG. 6. 5-consecutive-day monitoring of salivary Beta Amyloid 1-42 level from 6 healthy human subjects. morning. The average 5-day variance of the subjects are 13.3%.

The following steps are proposed: (a) expand the size of saliva testing pool (having different genders, age variations, life style variation, etc), (b) expand the AD biomarkers tested beyond Aβ-42 (tau, ApoE, BNP, etc) for better diagnosis accuracy, and (c) in collaboration with National Alzheimer's disease Centers, get the saliva from the AD patients, test AD biomarkers using D2PA assay, and compare with their CSF test and clinical tests. These studied will provide solid evidence if the Aβ-42 and other protein markers in saliva can be used in early detection of AD.

4. Noninvasive Early detection of breast cancer. CA15.3 is a tumor marker associated with mammary tumors. Increased levels of CA15.3 in serum have been observed in patients with breast cancer. It has been clinically approved to use CA15.3 for the monitoring, prognosis, and early detection of cancer recurrence. High elevated level of CA15.3, can provide valuable information for the early detection of the disease. Use of saliva is much simpler than serum and can be administrated by patients themselves. Compared with <30 U/mL in serum, CA15.3 in saliva for healthy human is <5 U/mL. Using the D2PA assay, the LoD was 0.001 U/mL, 5,000× more sensitive than previous assays, which is more than sufficient to identify CA15.3 in saliva. The use of the D2PA assay in measuring CA15.3 in healthy human will be investigated to validate CA15.3 in saliva, and then test CA15.3 in the saliva from cancer patients, and compare with other tests to validate D2PA in cancer early diagnosis.

5. Smart-phone based diagnosis assays for personalized medicine. The hardware and software for reading an assay using a smart phone will be developed, and the limit of detection (LoD) allowed by such approach will be determined (FIG. 1: Smart-phone based detection of fluorescence immunoassay on D2PA chips). The new ultra-sensitive assay platform technology will allow many diseases/cancer and other health related tests to be performed by smart-phone. In hardware, dipstick (self-pumping and multiplexed agents) will be designed and fabricated, and LED lighting and filters will be added. Software to control the reading and data analysis will be written. Initially simple fluorophors will be used in the test.

6. Further improve the assay technology, particularly even higher sensitivity and faster speed. The D2PA sensitivity, precision, linearity and repeatability will be improved by (i) optimizing the design of the D2PA (e.g. nanopillar size, pillar heights, nanodot size, nanogaps, metal used, other coupling layer) and (ii) using different fluorescence measurement methods (e.g. area-integrated measurement vs. pixel counting).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for monitoring the health condition of a subject, the method comprising:
i) processing an output from a signal enhancing detector, wherein the signal enhancing detector is configured to provide an output that is representative of a sample provided by a subject; and
ii) generating a report based on the processed detector output, wherein the report is related to health conditions of the subject.

2. The method according to claim 1, wherein the signal enhancing detector comprises a disk-coupled dots-on-pillar antenna array (D2PA).

3. The method according to claim 1, wherein the processing step i) comprises:
acquiring an image of a signal enhancing detector, wherein the signal enhancing detector is configured to indicate an output comprising a light signal that is representative of a sample provided by a subject; and
processing the light signal produced by the signal enhancing detector.

4. The method according to claim 3, wherein the light signal is a fluorescent or luminescent signal.

5. The method according to claim 1, wherein the method further comprises:
iv) receiving the report.

6. The method according to claim 1, wherein the report provides an advice to the subject based on the processed detector output.

7. The method according to claim 1, wherein the method comprises before step i):
applying a sample from a subject to a signal enhancing detector configured to produce an output that is representative of the sample.

8. The method according to claim 1, wherein the method further comprises:
delivering a report to the subject who holds the device or is in the same location of the device, and/or is in a remote location.

9. The method according to claim 1, wherein the method further comprises:
transmitting the processed detector output to a remote location where the transmitted information is analyzed; and
receiving the results of the analysis.

10. The method according to claim 1, wherein the analysis is done by software or by a professional.

11. The method according to claim 10, wherein the detector output further comprises an identifier for the signal enhancing detector.

12. The method according to claim 1, wherein the device is a hand held device.

13. The method according to claim 12, wherein the hand held device is a mobile phone.

14. A system comprising:
a device configured to:
acquire an output from a signal enhancing detector;
process the output to generate a report based on the processed output; and
provide the report to a subject,
wherein the signal enhancing detector is configured to obtain a sample provided from the subject and produce the output that is representative of the sample.

15. The system according to claim 14, wherein the signal enhancing detector comprises a D2PA.

16. The system according to claim 15, wherein the device is a hand held device.

17. The system according to claim 16, wherein the hand held device is a mobile phone.

18. A kit for monitoring the health status of a subject, the kit comprising:
a signal enhancing detector; and
instructions for performing the method of claim 1, wherein the device is a hand held device.

19. The kit according to claim 18, wherein the kit further comprises a computer-readable medium comprising software for implementing the method of claim 1 using a hand held device.

20. The kit according to claim 19, wherein the hand held device is a mobile phone.
